(19) United States
(12) Patent Application Publication
(10) Pub. No.: US 2010/0112630 A1
(43) Pub. Date: May 6, 2010

(54) METHODS FOR MEASURING MICROBIOLOGICAL CONTENT IN AQUEOUS MEDIA

(76) Inventors: Scott Martell BOYETTE, New Hope, PA (US); Hong Cai, Shanghai (CN); Paul Ronald Hirst, Redland Bay (AU); Yan Jin, Shanghai (CN); Juan Jiang, Shanghai (CN); Jie Li, Shanghai (CN); Rong Xu, Shanghai (CN); Kechao Yang, Shanghai (CN)

Correspondence Address: General Electric Company;GE Global Patent Operation, 2 Corporate Drive, Suite 648, Shelton, CT, 06484, US

(21) Appl. No.: 12/263,829

(22) Filed: Nov. 3, 2008

(51) Int. Cl. C12Q 1/06; G01N 33/00

(52) U.S. Cl. 435/39; 436/63

(57) ABSTRACT

A process for measuring total microbiological content in an aqueous medium includes adding a fluorescent dye to the aqueous medium, measuring the fluorescent signal in the aqueous medium to obtain a baseline fluorescent signal, releasing intracellular content of the microbiological matter into the aqueous medium by lysing the microbiological matter, measuring the fluorescent signal in the aqueous medium with the released intracellular content of the microbiological matter to obtain a second fluorescent signal, subtracting the baseline signal from the second fluorescent signal to obtain a net fluorescent signal and equating the net fluorescent signal with a microbiological content. Methods for measuring biofilm and adjusting for background noise are also provided.

### FIELD OF THE INVENTION

This invention relates to methods for quantifying microbiological content in aqueous media and more particularly, to fluorescence-based assays for measuring total microbiological content.

### BACKGROUND OF THE INVENTION

The presence of microbial activity in public water systems can cause health risks. Furthermore, detection and control of microorganisms in industrial systems is critical to various businesses, because the presence of such organisms contributes significantly to system corrosion, deposition and fouling and directly impacts the operation costs of the systems. Monitoring microbial concentrations in industrial systems and public water systems, and treatment of these systems, such as by the application of biocides, is an important part of maintaining these systems.

Conventional monitoring systems for microbial detection use culture-based methods or biochemluminescence-based methods. Both of these methods quantify microbial population; however, there are intrinsic shortcomings and defects affiliated with both of these methods. The culture-based method requires lengthy incubation time and often underestimates the microbial numbers due to the composition of the incubation medium. The biochemluminescence method is fast, but has poor accuracy and false positive and false negative results are frequently obtained.

Biofilms present additional concerns for monitoring microbial concentrations. Biofilms are groups of microbes that grow in complex aggregations and adhere to inert or living surfaces. Cells in a biofilm are held tightly to each other by a matrix of polymeric compounds, such as exopolysaccharides, lipopolysaccharides or glycoproteins. In addition to the fouling, corrosion problems and health concerns noted above, biofilms can reduce heat transfer and hydraulic pressure in industrial cooling water systems, plug water injection jets and clog water filters, and result in microbial influenced corrosion. Biofilms are protected by layers of expolymers and are extremely resistant to disinfectants and other biocides.

What is needed is an accurate and rapid method having a high degree of sensitivity for quantifying microbiological content, including quantifying biofilm content, in aqueous media.

### SUMMARY OF THE INVENTION

In one embodiment, a process for measuring total microbiological content in an aqueous medium including adding a fluorescent dye to the aqueous medium, measuring the fluorescent signal in the aqueous medium to obtain a baseline fluorescent signal, releasing intracellular content of the microbiological matter into the aqueous medium by lysing the microbiological matter, measuring the fluorescent signal in the aqueous medium with the released intracellular content of the microbiological matter to obtain a second fluorescent signal, subtracting the baseline signal from the second fluorescent signal to obtain a net fluorescent signal and equating the net fluorescent signal with a microbiological content.

The various embodiments provide improved methods for measuring total microbiological content in aqueous media, which are easy to use, inexpensive and accurate with a high degree of sensitivity and can be completed in a short period of time.

### BRIEF DESCRIPTION OF DRAWINGS

FIG. 1 depicts a graph of a regression plot of LOG delta RLU versus LOG cell concentration (cfu/ml) for Pseudomonas fluorescens diluted in autoclaved phosphate buffer saline (PBS).

FIG. 2 depicts a graph of a regression plot of LOG delta RLU versus cell concentration (cfu/ml) for Pseudomonas fluorescens diluted in filtered cooling tower water.

FIG. 3 depicts a graph of assay readings for cell concentration (cfu/ml) based on total microbiological content and plate count and ATP bioluminescence versus cell dilutions for Pseudomonas fluorescens diluted in autoclaved phosphate buffer saline (PBS).

FIG. 4 depicts a graph of assay readings for cell concentration (cfu/ml) based on total bacterial assay and plate count and ATP bioluminescence versus cell dilutions for Pseudomonas fluorescens diluted in filtered cooling tower water.

FIG. 5 depicts a graph of a regression plot of LOG delta delta RLU versus LOG cell concentration (cfu/ml) for Pseudomonas fluorescens diluted in autoclaved cooling tower water.

FIG. 6 depicts a graph of a regression plot of LOG delta RLU versus LOG cell concentration (cfu/ml) for Pseudomonas aeruginosa biofilm suspended in 0.85% saline buffer.

### DETAILED DESCRIPTION OF THE INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

In one embodiment, a process for measuring total microbiological content in an aqueous medium including adding a fluorescent dye to the aqueous medium, measuring the fluorescent signal in the aqueous medium to obtain a baseline fluorescent signal, releasing intracellular content of the microbiological matter into the aqueous medium by lysing the microbiological matter, measuring the fluorescent signal in the aqueous medium with the released intracellular content of the microbiological matter to obtain a second fluorescent signal, subtracting the baseline signal from the second fluorescent signal to obtain a net fluorescent signal and equating the net fluorescent signal with a microbiological content.

The process measures total microbiological content in an aqueous medium. The microbiological matter may be microbes, such as bacteria. Non-limiting examples of bacteria include Pseudomonas aeruginosa, Pseudomonas fluorescens, Pseudomonas putida, Desulfovibrio desuluricans, Klebsiella, Comamonas terrigena, Nitrosomonas europaea, Nitrobacter vulgaris, Sphaerotilus natans, Gallionella species, Mycobacterium terrae, Bacillus subtilis, Flavobacterium breve, Salmonella enterica, Enterica serovar Typhimurium, Bacillus atrophaeus spore, Bacillus megaterium, Enterobacter aerogenes, Actinobacillus actinomycetemcomitans, Candida albicans and Ecsherichia coli.

Aqueous medium may be any type of aqueous media that may contain microbiological matter including aqueous media into which biofilm microbes have been dislodged or dispersed. In one embodiment, the aqueous medium is water. In one embodiment, the water may be municipal water or industrial water, such as cooling tower water. In another embodiment, the aqueous medium may be aqueous solutions for personal care product manufacturing or food and beverage or pharmaceutical processing. In one embodiment, the aqueous media may be a saline solution. In another embodiment, the aqueous media may be a phosphate buffer solution.

A fluorescent dye is added to the aqueous medium. The fluorescent dye may be any type of dye that changes its fluorescence signal in the presence of microbiological matter. In one embodiment, the fluorescent dye is a fluorochrome, which is a microbiological staining dye that binds with biological cellular components, such as nucleic acids, proteins, cytoplasmic components and membrane components.

Examples of fluorochromes include, but are not limited to, acridine orange, ethidium bromide, Hoechst 33258, Hoechst 33342, propidium iodide, 4′,6-diamidino-2-phenylindole and nucleic acid dyes available commercially, such as PicoGreen®, SYTO® 16, SYBR Green I, SYBR® Green II, SYBR® Gold, YOYO™, TOTO™, TO-PRO®, YO-PRO®, Texas Red®, Redmond Red®, Bodipy® Dyes or Oregon Green®. Fluorochromes are commercially available from Molecular Probes (Eugene, Oreg.), Sigma Chemical (St Louis, Mo.), Amersham (Arlington Heights, Ill.), Callbiochem-Novabiochem (La Jolla, Calif.) or Synthetic Genetics (San Diego, Calif.). In another embodiment, the fluorochrome dye may be a cyanine dye, which is available commercially as PicoGreen®, TOTO™, SYBR® Green I, SYBR® Green II, SYBR® Gold or SYBR® Green I. In another embodiment, fluorochrome dye is an asymmetrical cyanine dye, such as SYBR® Green I.

The fluorescent dye is added to the aqueous medium in an amount suitable for fluorescing the microbiological matter in the aqueous medium. In one embodiment, the fluorescent dye is added in an amount of from about 0.5 mg to about 100 mg fluorescent dye per liter of aqueous medium. In another embodiment, the fluorescent dye is added in an amount of from about 0.5 mg to about 10 mg per liter of aqueous medium. In another embodiment, the dye is added in an amount of from about 0.5 mg to about 1.0 mg per liter of aqueous medium.

In one embodiment, a portion of the aqueous medium is removed for testing. Portions of the aqueous medium may be removed manually or may be removed systematically by an online testing device. The fluorescent dye is added to the aqueous medium and dispersed by mixing. In another embodiment, a solution of the fluorescent dye is injected into the aqueous medium sample and blended.

When using a fluorochrome, the pH of the aqueous medium is maintained within a suitable range for optimizing the fluorescence of the dye. In one embodiment, the pH of the aqueous medium is maintained from about 4.0 to about 9.5. In another embodiment, the pH of the aqueous medium is maintained from about 7.0 to about 8.0.

In one embodiment, a buffer is added to the aqueous medium to maintain the pH of the aqueous medium within a suitable range. The buffer may be any type of buffer that does not affect the microbiological matter or fluorescence measurements in the aqueous medium. In one embodiment, the buffer is an inorganic buffer, such as phosphate buffered saline or borate buffer. In another embodiment, the buffer is an organic buffer, such as tris(hydroxymethyl)aminomethane, ethylenediaminetetraacetic acid, N-2-hydroxyethylpiperazine-N′-2-ethanesulfonic acid or mixtures thereof. In one embodiment, the buffer is a blend of tris(hydroxymethyl)aminomethane and ethylenediaminetetraacetic acid. In another embodiment, a blend of tris(hydroxymethyl)aminomethane in a concentration range of about 1 mol/L to about 30 mmol/L and ethylenediaminetetraacetic acid in a concentration range of about 100 mmol/L to about 3 mmol/L is in a molar ratio of about 10:1.

The buffer may be added before or after the fluorochrome is added to the aqueous medium. In one embodiment, the fluorochrome and buffer are premixed and added together to the aqueous medium.

In one embodiment, the buffer is added to the aqueous medium in an amount of from about 1 percent by volume to about 30 percent by volume based on the volume of the aqueous medium. In another embodiment, the buffer is added to the aqueous medium in an amount of from about 1 percent by volume to about 15 percent by volume based on the volume of the aqueous medium. In another embodiment, the buffer is added to the aqueous medium in an amount of from about 5 percent by volume to about 10 percent by volume based on the volume of the aqueous medium.

A baseline fluorescent signal is obtained by measuring the fluorescence of the aqueous medium with the fluorescent dye. As used herein, “fluorescent” means the light emitted by a compound when excited by a shorter wavelength light. The excitation and emission wavelengths depend on the fluorescent dye selected. In one embodiment, the excitation wavelength is from about 350 nm to about 600 nm and the emission wavelength is from about 450 nm to about 650 nm.

Fluorescence may be measured by any type of fluorescence detector. In one embodiment, the fluorescent signal is measured by fluorescence spectroscopy, fluorescence microscopy, fluorescence diode array detection, micro plate fluorescence reading or flow cytometry. In one embodiment, the fluorescence detector is a portable fluorescence-based detection device or an online water condition monitoring instrument having fluorescence spectroscopy. In one embodiment, the portable fluorescence-based detection device has an LED excitation light and a PMT emission detector. In one embodiment, the portable fluorescence-based detection device has an LED excitation light and a photodiode emission detector.

The measurement is performed rapidly and several measurements may be taken and averaged. Microbiological matter may be detected at a concentration as low as 10⁴ colony forming units (cfu) per milliliter of aqueous medium tested without requiring a pre-test concentration process.

The baseline measurement can be recorded manually or is measured and stored in an online monitoring instrument.

The fluorescent dye stains microbiological cellular components, but cannot permeate in-tact cell membranes of the microbiological cells. To measure total microbiological content, the intracellular content of the microbiological matter is released into the aqueous medium where it can be contacted by the fluorescent dye. In one embodiment, the intracellular contents of microbiological matter is released by lysing cells of the microbiological matter, which breaks apart the cell membrane. Lysing may be performed using mechanical, chemical, physical, electrical, ultrasonic or microwave methods or any combination of these methods.

Mechanical lysing physically disrupts the cell barriers, such as by shear, vibration or force. Examples of mechanical methods include, but are not limited to, pressure-driven cell flow through filter-like structures or small scale bars in fluidic channels, osmotically stressing cells with rapid diffusional mixing of low ionic-strength water, subjecting cells to shear forces while entering a special region with sharp small-scale structures, disrupting cell barriers with a minibead beater or bead mill or applying ultrasonic energy to the cells in the aqueous medium.

Chemical lysing occurs when chemicals are used to disrupt the cell barriers and allow the intracellular content to be released Any chemical may be used that can disrupt the cell barriers. In one embodiment, detergents, enzymes, extraction solvents or lysing buffers are used. Detergents include, but are not limited to, dodecyl sulfate, 3-[(3-cholamidopropyl)dimethylammonio]-1-propanesulfonate, TWEEN™ 20 detergent, TRITON™ X series detergents, sodium cholate, sodium deoxycholate, guanidinium chloride. Enzymes include, but are not limited to, lysozymes, mutanolysin, labiase, lysostaphin, lyticase, proteinase K, endolysin or achromopeptidases. Extraction solvents include, but are not limited to, polyvinylpolypyrrolidone, phenol, trichlorotrifluoroethane or a mixture of phenol and guanidinium thiocyanate or guanidinium chloride. Lysing buffers include, but are not limited to, ammonium chloride, quaternary ammonium compounds, hexadecyltrimethylammonium bromide, cetyltrimethylammonium bromide, sodium dodecyl sulfate, hexametaphosphate, sodium pyrophosphate, Zap-o-globin™, a lysing buffer available commercially from Coulter Diagnostics or CyQUANT™ cell lysis buffer, available commercially from Molecular Probes.

The reagent may be added in any amount suitable for lysing the microbiological matter and may be added in excess. In one embodiment, the reagent is added in an amount of from about 1 mg to about 10,000 mg per liter of aqueous medium. In another embodiment, the reagent is added in an amount of from about 1 mg to about 1000 mg per liter of aqueous medium. In another embodiment, the reagent is added in an amount of from about 1 mg to about 50 mg per liter of aqueous medium.

Physical lysing may occur thermally or by freeze-thawing. Cell lysing can be accomplished thermally by heating the aqueous medium, such as with a thermal block or hot plate. In one embodiment, the aqueous medium is heated to a temperature from about 40° C. to about 100° C. In another embodiment, the temperature is from about 40° C. to about 60° C. In one embodiment, the aqueous medium is heated from about 1 minute to about 1 hour. In another embodiment, the aqueous medium is heated from about 1 minute to about 30 minutes, including from about 1 minute to about 15 minutes. In another embodiment, the aqueous medium is heated from about 1 minute to about 3 minutes.

In one example of freeze-thawing, the aqueous medium is frozen, such as in an ethanol-dry ice bath, and then thawed.

Cells may be lysed electrically with a series of electrical pulses, by diffusive mixing and dielectrophoretic trapping or by microwave radiation. Free radicals may also be used for cell lysing. The method includes applying an electric field to a mixture of a metal ion, peroxide and the microbiological matter in the aqueous medium to generate free radicals, which attack the cell barriers.

The fluorescent signals of the aqueous medium are measured before and after the intracellular content of the microbiological matter has been extracted and released into the aqueous medium to provide a baseline fluorescent signal and a second fluorescent signal, respectively. These fluorescent signals may be recorded manually or may be measured and stored in an online monitoring instrument.

The baseline fluorescent signal is subtracted from the second fluorescent signal to obtain a net fluorescent signal.

The net fluorescent signal may be equated with a total microbiological content. A calibration curve may be prepared for a selected fluorescent dye from known concentrations of microbiological matter and fluorescence measurements of the concentration. In one embodiment, the concentrations of microbiological matter are determined by plate count method. In one embodiment, several samples containing known total microbiological contents and the selected fluorescent dye are measured to obtain fluorescent signals. The log numbers of these signals are plotted on a graph and regression analysis may be performed to obtain a calibration curve equating total microbiological content with fluorescent signals.

Total bacterial concentration can be measured quickly and depending on the method selected for releasing extracellular contents of the biological matter, assays can be completed within 5 minutes. The rapid assays are well-suited to laboratory use, field applications, on-line automated batch systems or off-line monitoring systems. In another embodiment, the assays may be automated and performed continuously.

In another embodiment, a background fluorescent signal may be obtained to remove background interference and improve the accuracy of measuring the microbiological content in an aqueous medium. A background signal may be obtained by measuring the fluorescence of any additional organic or non-cellular components. In one embodiment, a background signal is subtracted from the net fluorescent signal. In one embodiment, a process for measuring total microbiological content in an aqueous medium includes adding a fluorescent dye to an aqueous medium portion, obtaining an additional aqueous medium portion for a background aqueous medium portion, treating the background aqueous medium portion to remove microbiological matter, adding a fluorescent dye to the treated background aqueous medium portion, measuring a fluorescent signal in the aqueous medium portion to obtain a baseline fluorescent signal, measuring a fluorescent signal in the treated background aqueous medium portion to obtain a background baseline fluorescent signal, releasing intracellular content of the microbiological matter in the aqueous medium portion into the aqueous medium by lysing the microbiological matter, simulating the lysing procedure in the background aqueous medium portion, measuring the fluorescent signal in the aqueous medium portion with the released microbiological intracellular content to obtain a second fluorescent signal, measuring the fluorescent signal in the simulated background aqueous medium portion to obtain a second background fluorescent signal, subtracting the baseline signal from the second fluorescent signal to obtain a net fluorescent signal, subtracting the background baseline fluorescent signal from the second background fluorescent signal to obtain a net background signal, adjusting the net fluorescent signal with the net background signal and equating the adjusted net fluorescent signal with a microbiological content.

The aqueous media is described above. Background signals may be obtained for any type of aqueous media, but are most helpful for aqueous media with high amounts of organics or non-cellular components that fluoresce in the presence of the fluorescent dye, such as process water from crude oil processing. In one embodiment, the aqueous medium portion and the background aqueous medium portion have the same volume.

Adding the fluorescent dye and steps for obtaining the baseline fluorescent signal, releasing the intracellular content of the microbiological matter, obtaining a second fluorescent signal and obtaining a net fluorescent signal are described above.

The aqueous medium may be treated to remove the microbiological matter. The microbiological matter may be removed from the aqueous medium for obtaining a background signal by heating the aqueous medium or by treating the aqueous medium with biocides, such as bleach, chlorine, other commercial biocides or combinations thereof. In one embodiment, chlorine is used in an amount of from about 0.1 ppm to about 30 ppm. In another embodiment, chlorine is used in an amount of from about 0.1 ppm to about 20 ppm, including from about 0.1 ppm to about 10 ppm. The biocide may be used in an amount of from about 1 ppm to about 200 ppm. In another embodiment, the biocide is used in an amount of from about 1 ppm to about 100 ppm, including from about 1 ppm to about 50 ppm. When using chlorine, it may be necessary to neutralize the chlorine after the background microbiological effect is minimized. In one embodiment, sodium meta bisulfite is used to neutralize the chlorine. In one embodiment, sodium meta bisulfite is added to the aqueous medium in an amount of from about 1 ppm to about 500 ppm. In another embodiment, sodium meta bisulfite is added to the aqueous medium in an amount of from about 1 ppm to about 300 ppm, including from about 1 ppm to about 200 ppm.

In another embodiment, the microbiological matter components may be removed by heating the aqueous medium, such as with a thermal block or hot plate. In one embodiment, the aqueous medium is heated to a temperature from about 40° C. to about 100° C. In another embodiment, the temperature is from about 40° C. to about 70° C. In another embodiment, the temperature is from about 40° C. to about 60° C. In one embodiment, the aqueous medium is heated from about 1 minute to about 1 hour. In another embodiment, the aqueous medium is heated from about 1 minute to about 30 minutes, including from about 1 minute to about 15 minutes. In another embodiment, the aqueous medium is heated from about 1 minute to about 3 minutes.

A background baseline fluorescent signal may be obtained by measuring the fluorescence of the aqueous medium portion that was treated to remove microbiological matter. The excitation and emission wavelengths depend on the fluorescent dye selected. In one embodiment, the excitation wavelength is from about 350 nm to about 600 nm and the emission wavelength is from about 450 nm to about 650 nm. Fluorescence may be measured by a fluorescence detector as described above. The background baseline signal can be recorded manually or is measured and stored in an online monitoring instrument.

The lysis procedure may be simulated in the treated background aqueous medium portion. In one embodiment, the process for releasing intracellular microbiological content into the aqueous medium portion is repeated in the background aqueous medium portion in which the microbiological matter has been removed. Lysing may be performed using mechanical, chemical, physical, electrical, ultrasonic or microwave methods or any combination of these methods, as is described above.

A second background fluorescent signal may be obtained by measuring the fluorescence of the simulated background aqueous medium. The excitation and emission wavelengths depend on the fluorescent dye selected. In one embodiment, the excitation wavelength is from about 350 nm to about 600 nm and the emission wavelength is from about 450 nm to about 650 nm. Fluorescence may be measured by a fluorescence detector, which are described above. The second background fluorescent signal can be recorded manually or is measured and stored in an online monitoring instrument.

The background baseline fluorescent signal may be subtracted from the second background fluorescent signal to obtain a net background signal. The net fluorescent signal may be adjusted by subtracting the net background signal from the net fluorescent signal to obtain an adjusted net fluorescent signal.

The adjusted net fluorescent signal may be equated with a total microbiological content. A calibration curve may be prepared for a selected fluorescent dye from known concentrations of microbiological matter and fluorescence measurements. In one embodiment, several samples containing known total microbiological contents and the selected fluorescent dye are measured to obtain fluorescent signals. The log numbers of these signals are plotted on a graph and regression analysis is performed to obtain a calibration curve equating total microbiological content with fluorescent signals.

Portions of the aqueous medium may be removed manually or may be removed systematically by an online testing device.

In another embodiment, the concentration of biofilm may be quantified. Biofilms cling to surfaces, including, but not limited to, glass, plastic, metal or paint, and can be dislodged from the surfaces and dispersed in an aqueous medium to measure the total microbiological content of the biofilm. In one embodiment, a process for measuring biofilm content in an aqueous medium includes dispersing biofilm into the aqueous medium, adding a fluorescent dye to the aqueous medium, measuring the fluorescent signal in the aqueous medium to obtain a baseline fluorescent signal, releasing intracellular content of the microbiological matter into the aqueous medium by lysing the microbiological matter, measuring the fluorescent signal in the aqueous medium with the released intracellular content of the microbiological matter to obtain a second fluorescent signal, subtracting the baseline fluorescent signal from the second fluorescent signal to obtain a net fluorescent signal and equating the net fluorescent signal with a microbiological content.

Biofilms or sessile microbes must be detached from surfaces and dispersed in an aqueous media to quantify the microbial concentration of the biofilms. Aqueous medium may be any type of aqueous media into which biofilm microbes have been dislodged or dispersed. In one embodiment, the biofilms are dispersed in a saline solution. In another embodiment, the biofilms are dispersed in a buffered saline solution. In another embodiment, the aqueous media may be a phosphate buffer solution. In another embodiment, the aqueous medium is water. In another embodiment, the water may be municipal water or industrial water, such as cooling tower water.

The microbial cells may be peeled or dislodged from the growth surface and dispersed into the aqueous medium by any suitable manner that does not disrupt the individual cell structure and may be achieved through a physical method, a mechanical method, a chemical method or a combination of these methods. Examples of physical methods for detaching and dispersing biofilm cells include, but are not limited to, agitation, vortexing, shaking and washing with strong shear stress. In one embodiment, the biofilm is dispersed with vortexing. In one embodiment, a biofilm coupon is submerged in a liquid and the cells are dislodged from the coupon by creating a flow of fluid that vortexes or swirls rapidly around as in a cyclone for a suitable time to release the cells from the aggregate. In one embodiment, the biofilm is vortexed for about 5 seconds to about 5 minutes. In another embodiment, the biofilm is vortexed from about 10 seconds to about 3 minutes. In another embodiment, the biofilm is vortexed from about 15 seconds to about 1 minute. In another embodiment, the biofilm is vortexed for about thirty seconds.

Examples of mechanical methods for detaching and dispersing biofilm cells include, but are not limited to, the use of a sonication bath or an electric current.

Examples of chemical methods for detaching and dispersing biofilm cells include, but are not limited to, adding a surfactant, dispersant or digestive enzyme. Examples of surfactants include, but are not limited to, ethylene oxide and/or propylene oxide (EO/PO) copolymers, dimethylamide polymer, Ultra-Kleen™ biocide, which is commercially available from Sterilex (Owings Mills, Md.), sodium octane sulfonate or alkyl polyglycoside. Examples of enzymes include, but are not limited to, blends of cellulase, alpha-amylase and protease. In one embodiment, the dispersant may be polyethyleneimine.

After the biofilm has been dislodged and dispersed in the aqueous medium, a total microbial assay is performed. The steps for adding a fluorescent dye to the aqueous medium, measuring the fluorescent signal in the aqueous medium to obtain a baseline fluorescent signal, releasing intracellular content of the microbiological matter into the aqueous medium, measuring the fluorescent signal in the aqueous medium with the released intracellular content of the microbiological matter to obtain a second fluorescent signal, obtaining a net fluorescent signal and equating the net fluorescent signal with a microbiological content are described above.

In another embodiment, the total amount of microbiology (cfu) may be obtained by multiplying the concentration with the known volume of aqueous media into which the biofilm was dislodged. In another embodiment, the amount of microbiology per surface unit area (cfu/cm²) may be obtained by dividing the amount of microbiology by the unit area of surface to which the biofilm was attached.

Biofilm can be measured directly by sampling biofilm from select system surfaces of known dimension. Alternatively, a coupon can be used to grow and measure the propensity of a system to grow biofilm. Some areas of water systems are inaccessible for practical sampling, and coupon testing provides a measure of the propensity for the system to grow biofilm. This method can also provide evidence that a treatment program has successfully reduced the propensity for the treated system to grow biofilm.

In another embodiment, a background fluorescent signal may be obtained to remove background interference and improve the accuracy of measuring the biofilm content in an aqueous medium.

In order that those skilled in the art will be better able to practice the present disclosure, the following examples are given by way of illustration and not by way of limitation.

### EXAMPLES

### Example 1

### Calibration Curve in Phosphate Buffer Saline (PBS):

Pseudomonas fluorescens cells were grown over night in a liquid culture media and added to 10 ml of PBS to form an initial sample. Serial dilutions were prepared from the initial sample. 0.1 ml of the initial sample was added to 9.9 ml of PBS to make a 1% (10⁻²) solution. 1 ml of the 1% solution was added to 9 ml of PBS to make a 0.1% (10⁻³) solution. 1 ml ofthe 0.1% solution was added to 9 ml of PBS to make a 0.01% (10⁻⁴) solution. 1 ml of the 0.01% solution was added to 9 ml of PBS to make a 0.001% (10⁻⁵) solution. 10 ml of the PBS was used for a cell-free blank

170 μl samples were taken from each of the diluted samples and the cell-free blank and each sample was mixed with 20 μl of 10× SYBR® Green I dye and 10 μl of 20× CyQUANT™ cell lysis buffer (available commercially from Molecular Probes). Fluorescence intensity was measured for each of the samples (cell-free blank, 10⁻², 10⁻³, 10⁻⁴ and 10⁻⁵) at an excitation wavelength of 497 nm and an emission wavelength of 520 nm by an LS55 Luminescence Spectrometer (PerkinElmer). The fluorescence was measured four times for each sample and averaged to obtain a Fluorescence Intensity I signal.

The samples were heated at 60° Celsius for 2 minutes and then cooled down to room temperature. Fluorescence intensity was measured for each of the diluted samples (10⁻², 10⁻³, 10⁻⁴ and 10⁻⁵) at an excitation wavelength of 497 nm and an emission wavelength of 520 nm. The fluorescence was measured four times for each sample and averaged to obtain a Fluorescence Intensity II signal.

A delta fluorescence intensity (Δ) was obtained by subtracting the Fluorescence Intensity I signal from the Fluorescence Intensity II signal.

Concentrations of the total Pseudomonas fluorescens bacteria were obtained for each sample (cell-free blank, 10⁻², 10⁻³, 10⁻⁴ and 10⁻⁵) using a standard plate count method.

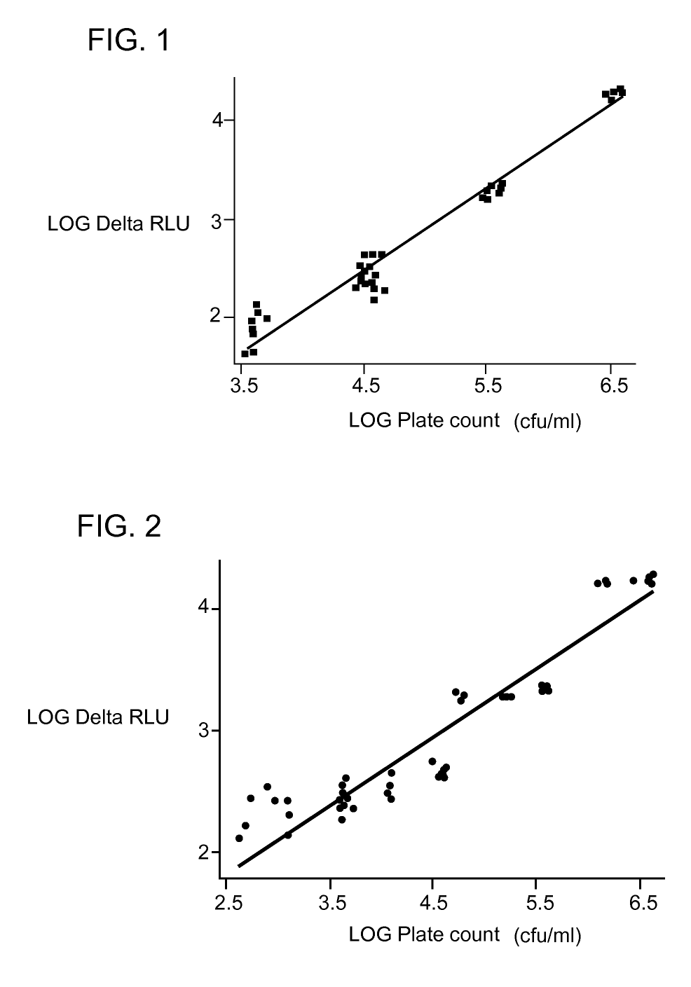

Regression analysis was performed between the log value of the delta fluorescence intensity (relative light unit (RLU)) and the log value of the plate count (cfu/ml) to obtain a calibration curve as shown in FIG. 1. The regression equation is y=−1.37+0.855×(R−Sq=97.6%).

### Example 2

### Calibration Curve:

A calibration curve was prepared as in Example 1 except that filtered water from a cooling tower was used instead of the PBS.

About 50 ml of water from a cooling tower was filtered through a PVDF filter (Millipore SLGV033RB) to remove residual microorganisms. 10 ml of the filtered water was used for a cell-free blank

Concentrations of the total Pseudomonas fluorescens bacteria were obtained for each sample (cell-free blank, 10⁻², 10⁻³, 10⁻⁴ and 10⁻⁵) by the plate count method.

Regression analysis was performed between the log value of the delta fluorescence intensity (RLU) and the log value of the plate count (cfu/ml) to obtain a calibration curve as shown in FIG. 2. The regression equation is y=0.383+0.576×(R−Sq=90.7%).

### Example 3

Pseudomonas fluorescens cells were grown over night on a culture plate and added to several 170 μl samples of phosphate buffer saline. Each sample was mixed with 20 μl of 10× SYBR® Green I dye (from Molecular Probes) and 10 μl of 20× CyQUANT™ cell lysis buffer.

Fluorescence intensity was measured for each of the samples at an excitation wavelength of 497 nm and an emission wavelength of 520 nm. The fluorescence was measured four times for each sample and averaged to obtain a fluorescent baseline signal.

The samples were heated at 60° Celsius for 2 minutes and then cooled down to room temperature. Fluorescence intensity was measured for each of the samples at an excitation wavelength of 497 nm and an emission wavelength of 520 nm. The fluorescence was measured four times for each sample and averaged to obtain a second fluorescent signal.

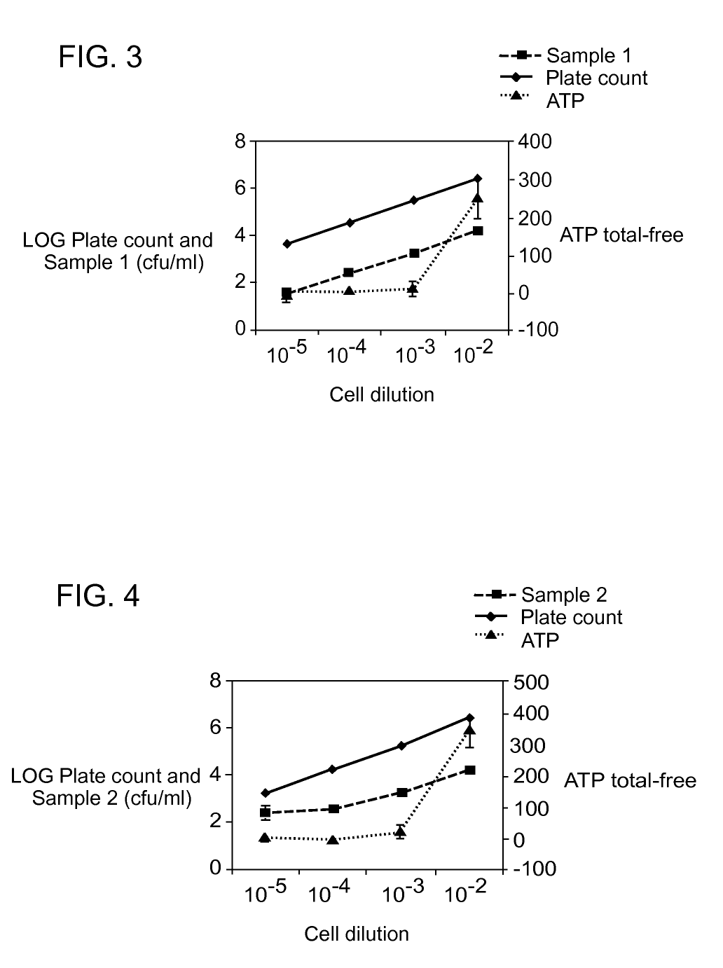

A delta fluorescence intensity (Δ) was obtained by subtracting the fluorescent baseline signal from the second fluorescent signal. The log value of the delta fluorescence intensity measurements were equated with a cell concentration (cfu/ml) from the calibration curve prepared in Example 1 and are shown as Sample 1 in FIG. 3. FIG. 3 depicts a graph of assay readings for cell concentration (cfu/ml) and ATP bioluminescence versus cell dilutions for Pseudomonas fluorescens diluted in phosphate buffer saline (PBS).

Comparative tests were also prepared on each sample by plate count and Bioscan™ ATP. Four measurements were prepared for each test and averaged and are shown in FIG. 3. Plate Count and the Sample 1 results are reported in log concentrations and ATP results are reported in original concentrations. ATP results had 1-log variance for the same standard and the results were too noisy to be used for quantitative comparisons.

Sample 1 was performed in 5 minutes or less and can measure concentrations as low as 10⁴ cfu/ml with good accuracy. It has a similar variation (standard deviation/mean) and good correlation with traditional culture-based methods, and has much better detection limit and smaller variation compared to the industrial Bioscan™ ATP method.

### Example 4

Pseudomonas fluorescens cells were grown over night on a culture plate and added to several 170 μl samples of field water that was autoclaved to remove residual microorganisms.

Each sample was mixed with 20 μl of 10× SYBR® Green I dye (from Molecular Probes) and 10 μl of 20× CyQUANT™ cell lysis buffer.

Fluorescence intensity was measured for each of the samples at an excitation wavelength of 497 nm and an emission wavelength of 520 nm. The fluorescence was measured four times for each sample and averaged to obtain a fluorescent baseline signal.

The samples were heated at 60° Celsius for 2 minutes and then cooled down to room temperature. Fluorescence intensity was measured for each of the samples at an excitation wavelength of 497 nm and an emission wavelength of 520 nm. The fluorescence was measured four times for each sample and averaged to obtain a second fluorescent signal.

A delta fluorescence intensity (Δ) was obtained by subtracting the fluorescent baseline signal from the second fluorescent signal. The log values of the delta fluorescence intensity measurements were equated with a cell concentration (cfu/ml) from the calibration curve prepared in Example 2 and are shown as Sample 2 in FIG. 4. FIG. 4 depicts a graph of assay readings for cell concentration (cfu/ml) and ATP bioluminescence versus cell dilutions for Pseudomonas fluorescens diluted in field water.

Comparative tests were also prepared on each sample by plate count and Bioscan™ ATP. Four measurements were prepared for each test and averaged and are shown in FIG. 4. Plate Count and the Sample 2 results are reported in log concentrations and ATP results are reported in original concentrations. The ATP results had 1-log variance for the same standard and the results were too noisy to be used for quantitative comparisons.

Sample 2 was performed in 5 minutes or less and can measure concentrations as low as 10⁴ cfu/ml with good accuracy. It has a similar variation (standard deviation/mean) and good correlation with traditional culture-based methods, and has much better detection limit and smaller variation compared to the industrial Bioscan™ ATP method.

### Example 5

Calibration curves were prepared for Pseudomonas fluorescens bacteria in cooling tower water and in phosphate buffer saline (PBS). About 50 ml water from a cooling tower was autoclaved to remove residual microorganisms.

Pseudomonas fluorescens cells were grown over night in a liquid culture media and added to 10 ml of the autoclaved cooling tower water to form an initial sample. Serial dilutions were prepared from the initial sample. 0.1 ml of the initial sample was added to 9.9 ml of autoclaved cooling tower water to make a 1% (10⁻²) solution. 1 ml of the 1% solution was added to 9 ml of autoclaved cooling water to make a 0.1% (10⁻³) solution. 1 ml of the 0.1% solution was added to 9 ml of autoclaved cooling water to make a 0.01% (10⁻⁴) solution. 1 ml of the 0.01% solution was added to 9 ml of autoclaved cooling tower water to make a 0.001% (10⁻⁵) solution. 10 ml of the autoclaved cooling tower water was used for a blank.

Pseudomonas fluorescens cells were added to 10 ml of the PBS to form an initial sample. Serial dilutions were prepared from the initial sample as for the cooling tower water to make PBS solutions of 10⁻², 10⁻³, 10⁻⁴ and 10⁻⁵. 10 ml of the PBS was used for a blank

A sample from each water and PBS serial dilution was set aside for measuring background noise in the water samples. Each background sample was treated with a biocide composed of 1 ppm chlorine and 20 ppm Bellacide® 350 for 30 minutes. 200 ppm sodium bisulfite was added to neutralize the residual chlorine.

170 μl samples were taken from each of the diluted cooling tower water PBS samples and background samples. Each sample was mixed with 20 μl of 10× SYBR® Green I dye (from Molecular Probes) and 10 μl of 20× CyQUANT™ cell lysis buffer.

Fluorescence intensity was measured for each of the cooling tower water and PBS samples at an excitation wavelength of 497 nm and an emission wavelength of 520 nm. The fluorescence was measured four times for each sample and averaged to obtain a Fluorescent I signal. Fluorescence intensity was measured for each of the background cooling tower water samples at an excitation wavelength of 497 nm and an emission wavelength of 520 nm. The fluorescence was measured four times for each sample and averaged to obtain a Background Fluorescent I signal.

The samples were heated at 60° Celsius for 2 minutes and then cooled down to room temperature. Fluorescence intensity was measured again for each of the cooling tower water and PBS samples at an excitation wavelength of 497 nm and an emission wavelength of 520 nm. The fluorescence was measured four times for each sample and averaged to obtain a Fluorescent II signal. Fluorescence intensity was measured for each of the background cooling tower water samples at an excitation wavelength of 497 nm and an emission wavelength of 520 nm. The fluorescence was measured four times for each sample and averaged to obtain a Background Fluorescent II signal.

A net fluorescence intensity was obtained by subtracting the Fluorescent I signal from the Fluorescent II signal. Net fluorescent measurements were obtained for each cooling tower water and PBS sample.

A net background fluorescent intensity was obtained by subtracting the Background Fluorescent Intensity I signal from the Background Fluorescent Intensity II signal. Net background fluorescent measurements were obtained for each background sample.

Adjusted net fluorescent signals were obtained by subtracting the net background fluorescent signal from the net fluorescent signal for each sample.

Concentrations of the total Pseudomonas fluorescens bacteria were obtained for each cooling tower water and PBS sample using a standard plate count method.

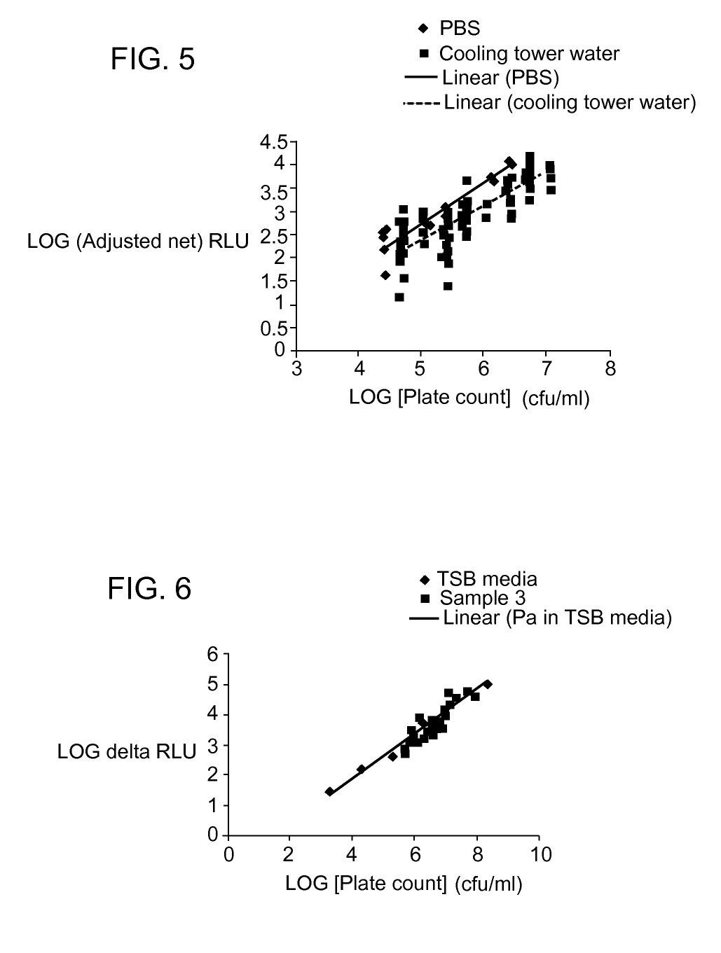

Regression analysis was performed between log value of the adjusted net fluorescent signal (RLU) and the log value of the plate count (cfu/ml) to obtain calibration curves for the cooling tower water and the PBS, as shown in FIG. 5. The regression equation for the PBS calibration curve is y=−1.47+0.847×(R−Sq=92.2%). The regression equation for the cooling tower water is y=−1.29+0.741×(R−Sq=73.7%). Three outliers out of 165 data points were deleted.

### Example 6

A calibration curve was prepared as in Example 1 except that the bacteria was Pseudomonas aeruginosa cells that were grown over night in a trypic soy broth (TSB) liquid culture media and added to 10 ml of 0.85% saline buffer to form an initial sample.

Serial dilutions were prepared from the initial sample. 0.1 ml of the initial sample was added to 9.9 ml of 0.85% saline buffer to make a 1% (10⁻²) solution. 1 ml of the 1% solution was added to 9 ml of 0.85% saline buffer to make a 0.1% (10⁻³) solution. 1 ml of the 0.1% solution was added to 9 ml of 0.85% saline buffer to make a 0.01% (10⁻⁴) solution. 1 ml of the 0.01% solution was added to 9 ml of 0.85% saline buffer to make a 0.001% (10⁻⁵) solution. 10 ml of the 0.85% saline buffer was used for a cell-free blank

180 μl were taken from each of the diluted samples and the cell-free blank and each sample was mixed with 20 μl of 10× SYBR® Green I dye. Fluorescence intensity was measured for each of the samples (cell-free blank, 10⁻², 10⁻³, 10⁻⁴ and 10⁻⁵) at an excitation wavelength of 497 nm and an emission wavelength of 520 nm by an LS55 Luminescence Spectrometer (PerkinElmer). The fluorescence was measured four times for each sample and averaged to obtain a baseline fluorescent measure.

The samples were heated to 90° C. for 2 minutes and then cooled to room temperature. Fluorescence intensity was measured at an excitation wavelength of 497 nm and an emission wavelength of 520 nm to obtain a fluorescent intensity II measurement. The fluorescence was measured four times for each sample and averaged to obtain a Fluorescent intensity II measurement.

A delta fluorescence intensity was calculated by subtracting the baseline fluorescent signal from the Fluorescent intensity II signal.

Concentrations of the total Pseudomonas aeruginosa cells were obtained for each sample (cell-free blank, 10⁻², 10⁻³, 10⁻⁴ and 10⁻⁵) using a standard plate count method.

Regression analysis was performed between the log value of the delta fluorescence intensity (RLU) and the log value of the plate count (cfu/ml) to obtain a calibration curve as shown in FIG. 6. The regression equation is y=−1.0185+0.7381×(R−Sq=98.97%).

Pseudomonas aeruginosa biofilm cells were grown over night on a 316 stainless steel tubing inner surface by providing a recycling flow of liquid growth media, 30% TSB media with 1% bacteria inoculum (over-night culture) through the tubing in a recycling circuit with a 135 ml/min flow rate.

A segment of the 316 stainless steel tube was removed from the flow system after a desired time interval. The biofilm build-up was dislodged by immersing the 316 stainless steel tube segment in 10 ml of 0.85% saline buffer and vortexed for 2 minutes at maximum speed.

Several aliquots of 180 μl of the vortexed sample were mixed with 20 μl of 10× SYBR® Green I dye. Fluorescence intensity was measured for each sample at an excitation wavelength of 497 nm and an emission wavelength of 520 nm. The fluorescence was measured four times for each sample and averaged to obtain a baseline fluorescent measurement.

The samples were heated to 90° C. for 2 minutes and then cooled to room temperature. Fluorescence intensity was measured for each of the samples at an excitation wavelength of 497 nm and an emission wavelength of 520 nm. The fluorescence was measured four times for each sample and averaged to obtain a fluorescent intensity II measurement.

A delta fluorescence intensity was calculated by subtracting the fluorescent baseline signal from the fluorescent intensity II signal. The log value of the delta fluorescent intensity measurements (RLU) were plotted along the calibration curve in FIG. 6 as Sample 3 data points. The log value of the delta fluorescent intensity measurements for each of the samples can be equated with a cell concentration (cfu/ml) from the calibration curve in FIG. 6.

From FIG. 6, it is can be seen that all the data points from the Pseudomonas aeruginosa biofilm cells (Sample 3) aligned well with the calibration curve obtained from the planktonic Pseudomonas aeruginosa cells suspension, which indicate this assay is suitable for biofilm quantification after dispersing the biofilm from the solid surface.

While typical embodiments have been set forth for the purpose of illustration, the foregoing descriptions should not be deemed to be a limitation on the scope herein. Accordingly, various modifications, adaptations and alternatives may occur to one skilled in the art without departing from the spirit and scope herein.

1. A process for measuring total microbiological content in an aqueous medium comprising adding a fluorescent dye to the aqueous medium, measuring the fluorescent signal in the aqueous medium to obtain a baseline fluorescent signal, releasing intracellular content of the microbiological matter into the aqueous medium by lysing the microbiological matter, measuring the fluorescent signal in the aqueous medium with the released intracellular content of the microbiological matter to obtain a second fluorescent signal, subtracting the baseline signal from the second fluorescent signal to obtain a net fluorescent signal and equating the net fluorescent signal with a microbiological content.

2. The method of claim 1 wherein the aqueous media comprises water, a saline solution or a phosphate buffer solution.

3. The method of claim 1 that wherein the fluorescent dye is a fluorochrome.

4. The method of claim 3 wherein the fluorochrome comprises acridine orange, ethidium bromide, Hoechst 33258, Hoechst 33342, propidium iodide, 4′,6-diamidino-2-phenylindole or a cyanine dye.

5. The method of claim 1 wherein the fluorescent dye is present in an amount of from about 0.5 mg to about 100 mg fluorescent dye per liter of aqueous medium.

6. The method of claim 3 wherein the pH of the aqueous medium is maintained from about 2 to about 10.

7. The method of claim 6, wherein a buffer is added to the aqueous medium to maintain the pH.

8. The method of claim 7 wherein the buffer is selected from the group consisting of phosphate buffered saline, borate buffer, tris(hydroxymethyl)aminomethane, ethylenediaminetetraacetic acid, N-2-hydroxyethylpiperazine-N′-2-ethanesulfonic acid and mixtures thereof.

9. The method of claim 1 wherein the fluorescent signal is measured with a fluorescent spectrometer at an excitation wavelength from about 350 nm to about 600 nm and an emission wavelength from about 450 nm to about 650 nm.

10. The method of claim 1 wherein the microbiological matter is chemically lysed with a lysing reagent to release intracellular material.

11. The method of claim 10 wherein the microbiological matter is lysed with a lysing reagent comprising detergents, enzymes, extraction solvents or lysis buffers.

12. The method of claim 10 wherein the lysing reagent is added in an amount of from about 1 mg to about 10,000 mg per liter of aqueous medium.

13. The method of claim 1 wherein the microbiological matter is physically, mechanically or electrically lysed to release intracellular material.

14. The method of claim 13 wherein the aqueous medium is heated to a temperature from about 40° C. to about 100° C. from about 1 minute to about 1 hour to lyse the cells of the microbiological matter.

15. The method of claim 1 wherein the net fluorescent signal is equated with a microbial concentration from a calibration curve.

16. The method of claim 15 wherein the calibration curve is prepared by measuring fluorescent signals for known concentrations of microbiological matter in aqueous media with the fluorescent dye, determining the net fluorescent signal for each concentration, plotting the concentration amounts versus log values of the net fluorescent signals on a graph and performing regression analysis to obtain the calibration curve.

17. The method of claim 26 wherein the known concentrations of microbiological matter are determined by plate count method.

18. The method of claim 1 further comprising adjusting the net fluorescent signal with a background signal.

19. The method of claim 18 wherein the method further comprises adjusting the net fluorescent signal with a background signal, said method further comprising obtaining an additional aqueous medium portion for a background aqueous medium portion, treating the background aqueous medium portion to remove microbiological matter, adding a fluorescent dye to the treated background aqueous medium portion, measuring a fluorescent signal in the treated background aqueous medium portion to obtain a background baseline fluorescent signal, simulating the lysing procedure in the background aqueous medium portion, measuring the fluorescent signal in the simulated background aqueous medium portion to obtain a second background fluorescent signal, subtracting the background baseline fluorescent signal from the second background fluorescent signal to obtain a net background signal, adjusting the net fluorescent signal with the net background signal and equating the adjusted net fluorescent signal with a microbiological content.

20. The method of claim 19 wherein the background aqueous medium portion is treated physically or chemically.

21. The method of claim 20 wherein the background aqueous medium portion is treated by heating the background aqueous medium at a temperature from about 40° C. to about 100° C. for about 1 minute to about 1 hour.

22. The method of claim 20 further comprising adding a biocide to the background aqueous medium.

23. A process for measuring total microbiological content in an aqueous medium includes adding a fluorescent dye to an aqueous medium portion, obtaining an additional aqueous medium portion for a background aqueous medium portion, treating the background aqueous medium portion to remove microbiological matter, adding a fluorescent dye to the treated background aqueous medium portion, measuring a fluorescent signal in the aqueous medium portion to obtain a baseline fluorescent signal, measuring a fluorescent signal in the treated background aqueous medium portion to obtain a background baseline fluorescent signal, releasing intracellular content of the microbiological matter into the aqueous medium portion by lysing the microbiological matter, simulating the lysing procedure in the background aqueous medium portion, measuring the fluorescent signal in the aqueous medium with the released microbiological intracellular content to obtain a second fluorescent signal, measuring the fluorescent signal in the simulated background aqueous medium portion to obtain a second background fluorescent signal, subtracting the baseline signal from the second fluorescent signal to obtain a net fluorescent signal, subtracting the background baseline fluorescent signal from the second background fluorescent signal to obtain a net background signal, adjusting the net fluorescent signal with the net background signal and equating the adjusted net fluorescent signal with a microbiological content.

24. The method of claim 1 wherein the microbial content comprises the content of biofilm and wherein the method further comprises dislodging and dispersing biofilm in the aqueous medium.

25. The method of claim 24 wherein the biofilm is dislodged physically by agitating or vortexing the aqueous medium.

26. The method of claim 24 wherein the biofilm is dislodged mechanically by using a sonication probe vibrating in the aqueous media.

27. The method of claim 24 wherein biofilm is dislodged from a surface when exposed to an electrical field.

28. The method of claim 24 wherein the biofilm is dislodged chemically by using a surfactant, biodispersant or a mixture thereof.

29. The method of claim 24 further comprising calculating the amount of microbiology per surface unit area to which the biofilm was attached.

30. The method of claim 24 further comprising adjusting the net fluorescent signal with a background fluorescent signal.

31. A method for measuring biofilm content in an aqueous medium includes dispersing biofilm into the aqueous medium, adding a fluorescent dye to the aqueous medium, measuring the fluorescent signal in the aqueous medium to obtain a baseline fluorescent signal, releasing intracellular content of the microbiological matter into the aqueous medium, measuring the fluorescent signal in the aqueous medium with the released intracellular content of the microbiological matter to obtain a second fluorescent signal, subtracting the baseline fluorescent signal from the second fluorescent signal to obtain a net fluorescent signal and equating the net fluorescent signal with a microbiological content.
